SEQUENCE LISTING (6 sequences)

SEQ ID NO: 1 - protein, 6 aa, Artificial Sequence
  Peptide
HHHHHH

SEQ ID NO: 2 - protein, 7 aa, Artificial Sequence
  Peptide
RFAACAA

SEQ ID NO: 3 - protein, 7 aa, Artificial Sequence
  Peptide
RFAALAA

SEQ ID NO: 4 - protein, 6 aa, Artificial Sequence
  Peptide
RAACAA

SEQ ID NO: 5 - protein, 6 aa, Artificial Sequence
  Peptide
RFACAA

SEQ ID NO: 6 - protein, 5 aa, Artificial Sequence
  Peptide
RFACA

(19) United States
(12) Patent Application Publication
(10) Pub. No.: US 2023/0181674 A1
(43) Pub. Date: Jun. 15, 2023

(54) ALLERGEN PROTECTION AGAINST URUSHIOLS

(71) Applicant: BEIERSDORF AG, Hamburg (DE)

(72) Inventors: Yvonne WOEHRMANN, Norderstedt (DE); Joern Hendrik REUTER, Henstedt Ulzburg (DE); Wilfried SIEFKEN, Hamburg (DE); Judith SEIDEL, Berlin (DE); Anna RINAS, Hamburg (DE); Marlis RIEDEL, Hamburg (DE); Tanja BUSSMANN, Hamburg (DE)

(21) Appl. No.: 17/759,636

(22) PCT Filed: Jan. 26, 2021
(86) PCT No.: PCT/EP2021/051719

§ 371 Date: Jul. 28, 2022

(30) Foreign Application Priority Data
Jan. 29, 2020 (DE) 10 2020 201 019.6

(51) Int. Cl. A61K 38/06; A61K 38/08

(52) U.S. Cl. CPC A61K 38/063; A61K 38/08

(57) ABSTRACT

Allergenic substances, allergens such as urushiols, are captured by peptides that consist of 2-10 amino acids, at least one amino acid having one or more reactive side chains. This barrier effect of the peptides, in particular in topically applicable preparations, leads to a novel anti-allergic form of therapy.

The invention is a preparation comprising specific peptides that can bind urushiol allergens, and the use of peptides to prevent or reduce the penetration or accumulation of urushiol allergens in or on the skin.

The skin forms a barrier that protects the human body from liquid or solid foreign substances. Nevertheless, foreign substances and thus potential allergens are still able to penetrate. This can trigger an immune response, causing the affected area of skin to become inflamed.

Normally, the immune system, which is made up of antibodies, white blood cells, mast cells, complement proteins, and other substances, defends the body against foreign substances (also termed antigens). On contact with certain substances (allergens) in the environment, food, or medicaments that are harmless to most people, the immune system of susceptible people can overreact. The result is an allergic reaction. Some people are allergic only to a single substance. Others in turn have an allergic reaction to many substances. About one quarter of all German citizens suffer from allergies.

Allergens can trigger an allergic reaction when coming into contact with the skin or eyes (or if they are inhaled, eaten, or injected). An allergic reaction can occur in different forms.

Penetrating substances, such as small chemical molecules, pollen, allergens, metal ions, but also proteases, can trigger pathologies such as atopic dermatitis or allergic contact dermatitis, which are triggered by reactions with molecular constituents of the skin or of the immune system.

At the present time, it is only the symptoms triggered by the penetration of foreign substances that are treated.

Allergens are substances that the body recognizes as foreign and that trigger an excessive immune response, an allergy. There are plant allergens such as pollen, animal allergens such as cat hair, and chemical allergens such as preservatives.

An allergen is a substance that can trigger hypersensitivity (allergic) reactions through the mediation of the immune system. An allergen is an antigen. Allergens have no chemical commonalities. It is therefore difficult to develop a chemical that is able to combat or destroy all allergens at the same time. Most allergens are proteins or protein compounds. On contact with allergens, the immune system of allergic patients responds by forming IgE antibodies. “Pseudoallergens”, on the other hand, are substances in which the immune system is not involved, but which are mediators, for example histamines.

Medicine distinguishes between four types of allergies, depending on which immunological response the allergen causes. The first three types of allergies are mediated by body fluids and by antibodies and other factors (humoral) contained therein; the fourth type of allergy proceeds on the other hand via a cell-mediated pathway (via T cells). There are however also mixed pictures.
Type I: Allergy of immediate type
Type II: Cytotoxic reaction
Type III: Immune complex formation
Type IV: Cellular immune response

Type I includes for example allergic rhinitis, nettle rash (urticaria) or else asthma symptoms, but also pollen allergies, insect venom allergies, and some drug or food allergies. Immediate-type allergic reactions often have a severe course. If a reaction to an allergen is particularly strong, the massive release of histamine can also trigger anaphylactic shock.

In type I allergy, the body forms corresponding group IgE (immunoglobulin E) antibodies immediately after contact with a specific allergen, the immune system having now been sensitized toward this antigen. The next time there is contact with an allergen, the IgE antibodies form a linkage to the so-called mast cells that are found in the skin and mucous membranes. They then bind the antigens to themselves. This bridging (between mast cell, antibody, and antigen) causes the mast cells to release the inflammatory mediator histamine (mast cell degranulation). The histamine and other substances released cause the uncomfortable symptoms typical of allergies. In the immediate vicinity, the histamine released causes blood vessels to dilate with the result that they become more permeable. The inflow of blood intensifies in order to be able to transport away foreign matter and waste. This leads to reddening and swelling of the skin or mucous membrane. Itching is also a frequent consequence of the inflammatory reaction.

Type IV, also termed delayed type, mainly triggers allergy symptoms on the skin, as is the case for example in nickel allergy. Contact allergies are generally responsible for a large proportion of cases resulting in incapacity for work.

Allergies of this type can develop unnoticed over years, but then suddenly trigger very violent immune reactions. In this case, foreign bodies that have penetrated are attacked directly by T-lymphocytes together with helper cells.

Contact allergens are triggers of type IV allergies. The typical clinical picture is allergic contact dermatitis, which appears on the exact same parts of the body that come into contact with the allergen concerned. Usually these are the hands, the face, the lower legs or the neck.

What is known is the group of plant contact allergens, the urushiols, which represent especially a major problem in the geographic range, for example in North America, of poison ivy, poison oak, and sumac, as representatives of the Anacardiaceae family.

Since there is no universal remedy against allergens, it would be desirable to prevent or at least reduce the penetration or accumulation of substances on or in the skin or the body.

Intercepting the active allergens and thus preventing the immune response would therefore be a preferred treatment.

The term peptide describes a molecule that is constructed from amino acids and which are linked to one another via peptide bonds. Peptides are systematically classified according to the number of amino acids from which they are constructed.

Thus, oligopeptides contain fewer than 10 amino acids, for example di- or tripeptides.

Polypeptides, on the other hand, contain a larger number of amino acids. Long polypeptide chains are also referred to as proteins.

In the condensation of amino acids, the carboxy group of one amino acid formally reacts with the amino group of the other amino acid to form the acid amide moiety —CO—NH—, with elimination of water. The newly formed amide bond between the carbon atom of the carbonyl group and the nitrogen atom thus becomes a peptide bond.

The 20 canonical amino acids are listed in the table below.

| Amino acid | | | Acyl group |
|---|---|---|---|
| Name | Abbrev. | Symbol | |
| Alanine | Ala | A | Alanyl- |
| Arginine | Arg | R | Arginyl- |
| Asparagine | Asn | N | Asparaginyl- |
| Aspartic acid | Asp | D | α-Aspartyl- |
| Cysteine | Cys | C | Cysteinyl- |
| Glutamine | Gln | Q | Glutaminyl- |
| Glutamic acid | Glu | E | α-Glutamyl- |
| Glycine | Gly | G | Glycyl- |
| Histidine | His | H | Histidyl- |
| Isoleucine | Ile | I | Isoleucyl- |
| Leucine | Leu | L | Leucyl- |
| Lysine | Lys | K | Lysyl- |
| Methionine | Met | M | Methionyl- |
| Phenylalanine | Phe | F | Phenylalanyl- |
| Proline | Pro | P | Prolyl- |
| Serine | Ser | S | Seryl- |
| Threonine | Thr | T | Threonyl- |
| Tryptophan | Trp | W | Tryptophyl- |
| Tyrosine | Tyr | Y | Tyrosyl- |
| Valine | Val | V | Valyl- |

In addition to the 20 proteinogenic amino acids, there are a whole series of other amino acids in nature that are referred to as non-proteinogenic amino acids.

There are also a number of amino acids that are chemically modified only after they have been incorporated into the growing polypeptide chain. These modified amino acids thus arise from proteinogenic amino acids by post-translational modification. One exception in some organisms is selenocysteine, in which the S atom in cysteine is replaced by selenium. Selenocysteine has its own tRNA and a codon that is otherwise regarded as a stop codon in the genetic code, which means that selenocysteine can in fact be counted as one of the proteinogenic amino acids. This amino acid occurs for example in the formate dehydrogenase of the bacterium E. coli and in mammalian glutathione peroxidase.

Homocysteine, L-homocysteine (Hcy), is also a naturally occurring but non-proteinogenic α-amino acid.

There are in nature 20 standard amino acids that, aside from a few exceptions, make up every protein. This accordingly places limits on the biochemical, biophysical, and reactive properties of desired functional proteins and peptides. However, there are now methods for introducing chemically modified amino acids having a wide variety of reactive groups in site-specific positions. A basis therefor achieved is for example the methodology of amber suppression or a coupling of reactive groups via click chemistry.

In cosmetics, peptides are used inter alia to improve skin characteristics. They are commonly used to reduce the depth of wrinkles. There are however also other possible uses, for example peptides that have a beneficial effect on the water content of the skin by improving the synthesis of the water channels in the skin.

WO 2012164488 A2 describes a mixture of a tripeptide and a tetrapeptide for therapeutic treatment of the papillary dermis and to prevent and/or treat skin aging, especially photo-induced aging.

EP 979829 A2 describes for example the use of oligopeptides for cosmetic and topical dermatological skin lightening or for preventing skin tanning.

Peptides are known to be susceptible to oxidation reactions and/or degradation reactions, which are made noticeable through discolorations or off-odors, which makes their incorporation and use in cosmetic preparations problematic.

Increasingly adverse environmental influences and a greater abundance of foreign substances are coming into contact with human skin and can lead to discomfort, skin irritation, and pathologies, as described above for allergens.

Besides allergies, foreign substances such as allergens and, in particular, urushiols can, however, also cause dermatologically relevant skin changes, chronic conditions such as atopic dermatitis.

It is therefore additionally desirable to provide a barrier against such foreign substances, in particular an allergen barrier.

It is also desirable that the barrier system is easy to apply, permits a broad range of applications, and is designed to be stable, straightforward to store and straightforward to use.

One embodiment of the invention comprises preparations for topical application comprising one or more peptides for preventing or attenuating allergic reactions in or on the skin. The allergic reactions are here caused by the penetration and/or accumulation of foreign substances and/or allergens such as urushiols on and/or in the skin. The peptides of the invention are characterized in that they contain 2 to 10 amino acids and that at least one of these amino acids has one or more reactive side chains.

A further embodiment of the invention is a preparation for topical application comprising one or more peptides for preventing or reducing the penetration and/or accumulation of foreign substances and/or allergens such as urushiols on and/or in the skin.

The peptides form a skin barrier for urushiols. The peptides thus result in particular in the prevention or attenuation of allergic reactions in or on the skin. Preference is thus also given in accordance with the invention to the prevention or attenuation of allergic contact dermatitis caused by urushiols by the peptides of the invention or by the preparations in which they are present.

One embodiment of the invention therefore comprises also peptides for use in the treatment of allergic contact dermatitis caused by urushiols.

The peptides of the invention can be used as scavengers against foreign substances such as urushiols.

The peptides of the invention advantageously bind contact allergens. A contact allergy is an allergy to mostly small-molecular-weight substances known as contact allergens. These commonly occur in everyday life or at the workplace. It is based on a T-cell-mediated specific response of the immune system (type IV/delayed-type sensitization) to reactive substances that, as so-called haptens, generally become an allergen only after binding to proteins in the skin. Known contact allergens include special natural substances (e.g., plant substances). Contact allergy is manifested clinically as allergic contact dermatitis. Contact dermatitis (also termed contact eczema) is an inflammatory, non-infectious intolerance reaction of the skin or mucous membranes caused by external contact with noxae that have an immunological, chemical or physical effect. The main triggers are allergens and irritant substances.

The peptides of the invention or the preparations in which they are present are therefore preferably used to prevent or reduce the penetration and/or accumulation of contact allergens on and/or in the skin.

The peptides form a skin barrier for the contact allergen. The peptides thus result in particular in the prevention or attenuation of allergic, in particular contact-allergic, reactions in or on the skin. Preference is thus also given in accordance with the invention to the prevention or attenuation of allergic contact dermatitis by the peptides of the invention or by the preparations in which they are present.

Preference is given to employing the peptides of the invention against chemical allergens in order in to prevent especially type I and especially type IV allergies.

Chemical allergens include for example textile fibers, dyes, coating substances or other chemical substances.

Preference is given to employing the use and method according to the invention against these substance classes.

Particular preference is given to using the peptides of the invention against chemically electronegative allergens.

The peptides of the invention may be employed with preference against urushiol contact allergens.

The contact allergens to be preferably prevented include plant substances, in particular urushiols.

Preference is in accordance with the invention thus given to preventing or attenuating the development of a contact allergy and/or contact dermatitis on contact with allergens.

A further preferred embodiment of the invention is also the non-therapeutic, in particular cosmetic use of one or more peptides or of the preparation in which they are present as a skin barrier against urushiols.

Another embodiment of the invention is a cosmetic method for protecting against urushiol allergens by applying to the skin a preparation comprising one or more peptides of the invention.

The non-therapeutic use and the cosmetic method of the peptides of the invention serve to prevent or reduce the penetration and/or accumulation of urushiols on and/or in the skin. Thus, a contribution to skin care is inter alia also made.

The peptides of the invention are selected from the group of peptides having 2-10 amino acids and are characterized in that they contain one or more amino acids having one or more reactive side chains.

The peptides of the invention preferably contain at least one amino acid selected from the group histidine, cysteine, lysine, tyrosine, serine, selenocysteine, homocysteine, asparagine, aspartic acid, glutamine, glutamic acid, methionine, phenylalanine and/or pyrrolysine.

Preference is in turn given to selecting peptides from the group GSH, HHHHHH, Ac-RFAACAA, Ac-RFAALAA, RFAALAA, RFAACAA, Ac-RAACAA, RAACAA, Ac-RFACAA, RFACAA, Ac-RFACA and/or RFACA.

The sequence listing of the peptides that are preferred according to the invention is:

Seq. 1: HHHHHH = His His His His His His

Seq. 2: RFAACAA = Arg Phe Ala Ala Cys Ala Ala

Seq. 3: RFAALAA = Arg Phe Ala Ala Leu Ala Ala

Seq. 4: RAACAA = Arg Ala Ala Cys Ala Ala

Seq. 5: RFACAA = Arg Phe Ala Cys Ala Ala

Seq. 6: RFACA = Arg Phe Ala Cys Ala

and in each case the acetylated acids thereof (Ac-).

For topical application, the peptides of the invention are preferably incorporated in cosmetic or dermatological preparations in order to achieve user-friendly topical application of the allergen barrier.

The invention is therefore also a preparation for topical application, in particular a cosmetic preparation, comprising one or more peptides from the group of peptides having 2-10 amino acids, wherein at least one of these amino acids has one or more reactive side chains.

The content of peptides of the invention having 2 to 10 amino acids is advantageously within a range from 0.01% to 10% by weight, especially within a range from 0.1% to 5% by weight, more preferably within a range from 0.2% to 5% by weight, based on the total mass of the preparation.

A method for protecting against urushiol allergens is thus possible by applying to the skin a preparation comprising one or more peptides, wherein the peptides are selected from the group of peptides having 2-10 amino acids and at least one of these amino acids has one or more reactive side chains.

According to the invention, the term “skin barrier” and “barrier effect” encompasses intercepting, blocking, inactivating, preventing, reducing, and/or hindering the penetration or accumulation of foreign substances such as allergens, in particular contact allergens, on or in the skin. The substances giving rise to an undesired reaction are in accordance with the invention collectively referred to in simple terms as foreign substances and/or allergens.

Attachment or binding of the allergen to the peptide prevents or at least reduces accumulation or penetration of the allergen on or in the skin.

A reduction in the accumulation of allergens on the skin takes place in respect both of the amount of allergen and the rate.

In other words, one or more peptides of the invention are used to intercept, block and/or inactivate allergens and/or to prevent, reduce, or hinder the penetration or accumulation of foreign substances or allergens on or in the skin, in particular human skin.

Peptides of the invention intercept the foreign substances, individual substances such as urushiols. They thus prevent, reduce or hinder the ability of these substances to accumulate on the skin or penetrate into the skin and to result there in undesirable reactions on or in the skin or the body. The triggering of an immune response is consequently prevented. More particularly, prevention of the penetration of allergens means that an allergic reaction is attenuated or prevented altogether.

More particularly, allergic contact dermatitis caused by urushiols is thus prevented.

The peptides of the invention are selected from the group of peptides having 2-10 amino acids. The peptides are preferably selected from the group of up to 8 amino acids, especially fewer than 8 amino acids.

Peptides having a high number of amino acids run the risk of being classed as a foreign substance when they come into contact with the skin, and possibly triggering a specific immune defense via MHC groups.

The abbreviation MHC stands for “major histocompatibility complex”, a group of very varied genes that play a role in the immune response of vertebrates. The genes provide the instructions for the construction of transporter molecules. Their role is to hold on to protein fragments (peptides) from viruses and bacteria and present them to specialized cells of the immune system: this allows them to recognize and fight the enemies.

The peptides of the invention are further characterized in that they contain one or more amino acids that in turn have one or more reactive side chains.

Reactive side chains are characterized in that they contain electron donors and/or acceptors. These properties make it possible for such side chains to be involved in the formation of covalent bonds, coordinate bonds, hydrogen bonds or van der Waals interactions between allergen/irritant/foreign substance and peptide.

The peptides of the invention advantageously contain at least one amino acid having a free thiol group. The free thiol group is a reactive side chain such as that found in for example the amino acid cysteine.

Thus, the peptide glutathione (GSH) or γ-L-glutamyl-L-cysteinylglycine are also for example preferred peptides having a reactive side chain. γ-L-Glutamyl-L-cysteinylglycine is a tripeptide having the sequence glutamic acid-cysteine-glycine (Glu-Cys-Gly).

In accordance with the invention, a side chain can also be referred to as a group and thus characterizes the reactivity and thus allergen binding of peptides containing it.

When peptides that initially contain no reactive group, for example no thiol group, are used according to the invention, they are converted, through addition of one or more amino acids having reactive groups, for example cysteine, into a peptide of the invention that has a reactive group. The designation of the peptides of the invention can in that case still include the original peptide designation, this being extended by the addition of the reactive amino acid(s).

Given the standard designation for peptides without a thiol group, for example oligopeptide-6, e.g., KDIFTRALN, adding the amino acid cysteine results in an extended peptide of the invention having the sequence CKDIFTRALN, or Cys-oligopeptide-6.

The peptides of the invention advantageously contain one or more particularly chemically active amino acids such as histidine, cysteine, lysine, tyrosine, serine, selenocysteine, homocysteine, asparagine, aspartic acid, glutamine, glutamic acid, methionine, phenylalanine or pyrrolysine.

It has been found that allergens show a high affinity for binding to these reactive amino acids. Surprisingly, these specific peptides are thus able to bind the penetrating substances and be used in accordance with the invention.

The binding takes place in a wide variety of ways, for example by intermolecular interactions, ionic bonds, hydrogen bonds, van der Waals bonds or covalent bonds. Overlapping of different binding modes can also occur.

What is key is purely the effect thereby achieved - the barrier effect - that results in inactivation of urushiol allergens, preventing them from penetrating into the skin or from accumulating on the skin.

By exploiting the affinity of allergens for the reactive amino acid side chains or groups, the use according to the invention opens up the means of preventing or attenuating an allergic skin reaction on contact with urushiol allergens that has long been sought.

A novel antiallergic agent/novel antiallergic method has according to the invention thus been created that does not eliminate or at least alleviate the symptoms of an allergic pathology, but rather prevents an allergic reaction from developing in the first place.

Examples of reactive amino acids are cysteine and methionine. Both contain a sulfur atom. Cysteine is a polar amino acid and has a free thiol group. This property of cysteine is very important for the structure of proteins, since it is how polypeptide chains are linked to one another or else covalent linkages are created within a polypeptide chain that considerably stabilize the conformation of a protein. The term cystine is sometimes also used for two cysteines connected via disulfide bridges. In metalloproteins, it is often a cysteine residue that binds zinc, copper, or iron atoms.

Hexahistidine is also important in the binding of metalloproteins.

This mode of action is also utilized in the coupling of nickel, thus classic nickel allergy is attenuated or even prevented altogether by applying a peptide of the invention.

The peptides of the invention can advantageously contain both naturally occurring amino acids and also amino acids synthetically produced and modified, for example by acetylation.

Examples of preferred peptides are

Glutathione (GSH), also known as γ-L-glutamyl-L-cysteinylglycine, is a tripeptide having the sequence glutamic acid-cysteine-glycine.
Hexahistidine (HHHHHH)
Ac-RFAACAA - a peptide having the sequence acetyl-arginine-phenylalanine-alanine-alanine-cysteine-alanine-alanine

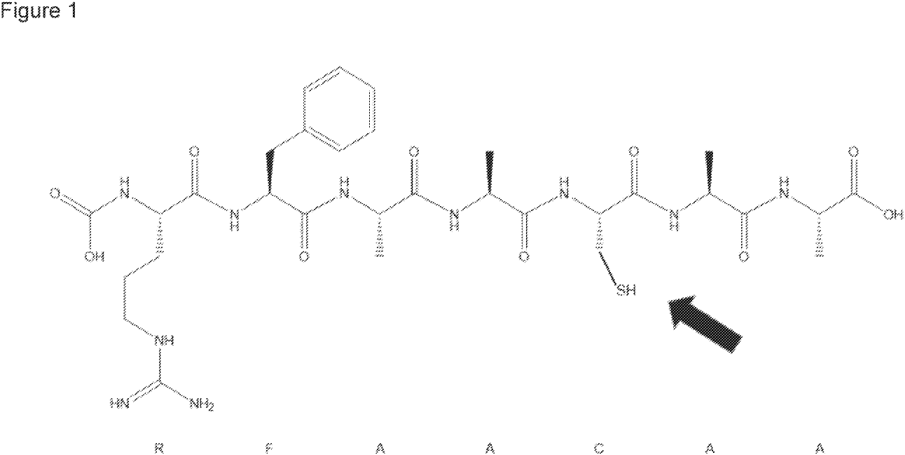

The peptide is shown by way of example in FIG. 1, in which the active amino acid side chain is marked with an arrow.
Ac-RFAALAAA - a peptide having the sequence acetyl-arginine-phenylalanine-alanine-alanine-lysine-alanine-alanine
Ac-RAACAA
Ac-RFACAA
Ac-RFACA
RFAALAA
RFAACAA
RAACAA
RFACAA
RFACA

The peptide sequence listing of these peptides is given in the appendix.

For use, one or more peptides of the invention comprising one or more active amino acids are applied to the surface of the skin.

The invention therefore also encompasses preparations for topical application, in particular cosmetic or dermatological preparations, comprising one or more of the peptides of the invention.

Topical application can take place in many different ways, for example in the form of a cosmetic formulation. Application to the skin results in the creation of a protective mechanism, a barrier effect.

Simple aqueous or lipid-containing formulations up to preparation forms with a complex make-up are possible, depending on the field of use.

The skin is in constant contact with foreign substances from the environment, or allergens such as urushiols. The protective mechanism means that foreign substances from the environment now come up against this barrier first. Potential allergens encounter the peptides contained in the applied formulation and are thus able to directly react with or be bound or at least hindered by the active amino acids of these peptides. The allergens are thus intercepted and are no longer able to cause a biological reaction in the body.

The allergen reaction with effector (skin’s own peptide or protein) is prevented before an allergy to the allergen can develop.

Likewise, the allergens are intercepted and the triggering of the allergy by the specific allergen (elicitation) thus prevented.

The barrier effect of the peptides of the invention and their prevention or attenuation of allergic reactions has been impressively demonstrated in the investigations elucidated below.

One operating principle in the experiments carried out here is based primarily on the scavenging of allergens through the formation of covalent bonds to the reactive group, for example the amino acid cysteine, according to the mechanism of a nucleophilic addition, also termed SN1 and SN2.

The following investigations were carried out to demonstrate the barrier effect.

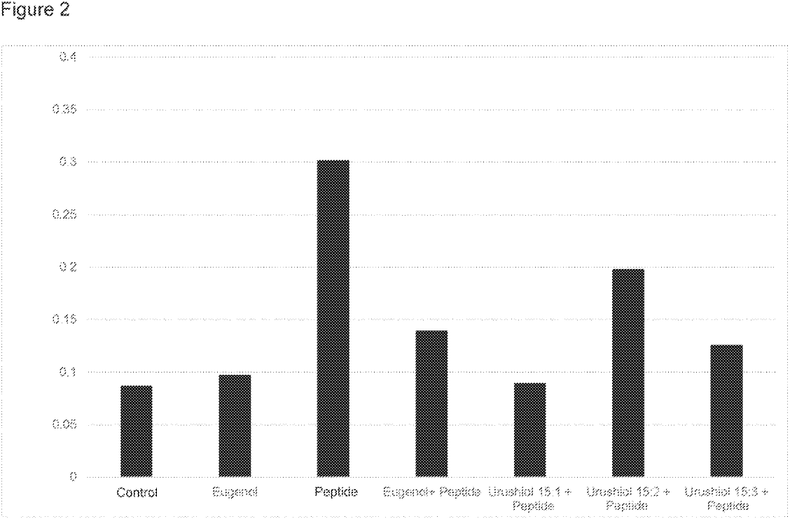

FIG. 2 shows investigations into the reactivity of peptides of the invention with urushiol allergens after 24 h. The absorbance is plotted on the y-axis. The peptide-allergen reactions were realized according to a direct peptide reactivity assay. The test is referred to as the in-chemico method, that is to say no cells are used, but a chemical reaction is carried out.

The direct peptide reaction assay (DPRA) is an OECD-validated test method.

The native, still-unbound peptide is detected by reaction with Ellman’s reagent and corresponding absorbance measurement. The direct peptide reactivity assay (DPRA) is a chemical method for predicting epidermal protein binding. DPRA uses for example HPLC to measure the depletion of peptides in solution after exposure to test chemicals.

RFAACAA was selected as an example of a peptide of the invention.

In the DPRA method, the absorbance at 415 nm is determined as the measured variable.

The allergen concentration is in the investigations carried out a standard 5 mM and thus 10 times the concentration of the peptide used (0.5 mM).

The reaction courses shown were determined colorimetrically through the reaction of the peptide with Ellmann’s reagent in a photometer. In this reaction, DTNB ((5,5′-dithiobis-2-nitrobenzoic acid, Ellmann’s reagent) is cleaved and attached to the SH residue of the amino acid cysteine of the peptides of the invention (RFAACAA).

The SH radical is at the same time the reactive binding site of the peptide with the allergens. The color reaction with DTNB (color change from colorless to yellow) competes with the allergen reaction. This means that the better the allergen reacts with the peptide, the less peptide remains available for the reaction with the detection reagent, i.e. the less the resulting yellow coloration.

FIG. 2 shows the allergen reduction (urushiols and model allergen eugenol) in the direct peptide reaction assay (DPRA) after a 24 h reaction, n = 3.

FIG. 2 shows the fall in peptide against eugenol and against various urushiol derivatives. The control is the peptide in the buffer system without allergen, i.e. instead of allergen only its solvent (ethanol) is added.

Urushiols of the structure below were used.

| | | |
|---|---|---|
| Urushiol I | 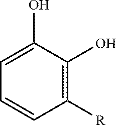 | —(CH₂)₁₄CH₃ |
| Urushiol II | | —(CH₂)₇CH═CH(CH₂)₅CH₃ |
| Urushiol III | | —(CH₂)₇CH═CHCH₂CH═CH(CH₂)₂CH₃ |
| Urushiol IV | | —(CH₂)₇CH═CHCH₂CH═CHCH═CHCH₃ |
| Urushiol V | | —(CH₂)₇CH═CHCH₂CH═CHCH₂CH═CH₂ |

The investigated urushiols differ in the saturation in the side chain R.

Urushiols where R = C15 singly (C15:1), doubly (C15:2) or triply (C15:3) unsaturated were investigated.

The results demonstrate very high reactivity and thus protective activity of the peptides of the invention against urushiols too. The peptides, especially Ac-RFAACAA and RFAACAA, show very high reactivity and thus protective activity against urushiol structures I-V in particular.

Urushiols are the strongest naturally occurring allergen, which is in addition responsible for many sensitizations, as explained above. Urushiols are the main sensitizing constituent of the sumac plants, such as poison sumac (Toxicodendron quercifolium), including, in particular, in the various Rhus species such as poison ivy (Toxicodendron radicans), poison oak (Toxicodendron diversilobum), and lacquer tree (Rhus verniciflua), and also in Toxicodendron rydbergii, Toxicodendron toxicarium, and Toxicodendron vernix.

Allergy to urushiols, which requires prior sensitization, can be triggered in approx. 50-75% of all Americans. This contact dermatitis affects 10-50 million Americans every year.

Contact allergy to urushiols is a serious occupational disease, for example in agricultural and forestry workers and firefighters, but also in all recreation seekers who move around in nature. In the USA they are responsible for 7.1 million doctor visits and 430000 hospital inpatient stays. For example, this contact allergy is the cause of 10% of all work time lost to injury at US Forest Services, and approximately one-third of forestry workers in California, Oregon, and Washington are unfit for work during the wildfire season because of these reactions.

The invention now provides a possible remedy here.

The preparations of the invention comprising the short-chain peptides of the invention result in alleviation of the allergic skin reaction to constituents of poison ivy.

All investigations impressively demonstrate the allergen barrier effect of the peptides of the invention in preventing or reducing the penetration and/or accumulation of urushiols on and/or in the skin.

The numerous investigations of the various peptides demonstrate by way of example the barrier effect and the prevention or attenuation of allergic reactions for all peptides of the invention having a total of 2 to 10 amino acids, wherein at least one amino acid has one or more reactive side chains.

The peptides of the invention can advantageously be incorporated into typical cosmetic and dermatological preparations, which can take different forms. For instance, examples of preferred preparation forms are a solution, a water-in-oil (W/O) type emulsion or an oil-in-water (O/W) type emulsion, or multiple emulsions, for example a water-in-oil-in-water (W/O/W) type or oil-in-water-in-oil (O/W/O) type, a hydrodispersion or lipodispersion, a gel, a solid stick or even an aerosol.

The content of one or more peptides of the invention in preparations for topical application is advantageously chosen within a range from 0.01% to 10% by weight, especially within a range from 0.1% to 5% by weight, more preferably within a range from 0.2% to 5% by weight, based on the total mass of the preparation.

Preferred application forms of the peptides of the invention are emulsion preparations, gels, and spray formulations.

Emulsions produced according to the invention, for example in the form of a cream, a lotion or a cosmetic milk, are advantageous and comprise, for example, fats, oils, waxes, and/or other fatty substances, and also water and one or more emulsifiers as are customarily used for formulations of this type.

The preparations of the invention advantageously comprise one or more film formers.

In addition, the peptides can be chemically modified to improve the physicochemical properties. For example, derivatization with fatty acid residues (for example acetyl, caproyl, undecenoyl, or palmitoyl) increases the lipophilicity. Secondly, chemical modifications can help make the peptide more stable and thus protect it for example from degradation by peptidases.

So-called penetration enhancers, solubilizers and physical methods are able to increase the permeability of the skin and also further boost distribution in the skin.

An addition of solubilizers is therefore advantageous.

It is of course known to those skilled in the art that sophisticated cosmetic compositions are generally not conceivable without the customary auxiliaries and additives. These include for example consistency enhancers, fillers, dyes, emulsifiers, additional active substances such as vitamins or proteins, light stabilizers, stabilizers, insect repellents, alcohol, water, salts, EDTA, antimicrobial, proteolytic or keratolytic substances, etc., the addition of potential allergens being of course preferably avoided.

Mutatis mutandis, corresponding requirements apply to the formulation of medicinal preparations.

Allergy-triggering substances, such as urushiols, are intercepted by the peptides of the invention consisting of 2-10 amino acids, wherein at least one amino acid has one or more reactive side chains. This barrier effect of the peptides, particularly in preparations for topical application, results in a novel form of antiallergic therapy.

### Example Preparations

The numerical values are contents by weight based on the total mass of the preparation.

| | 1 | 2 | 3 | 4 |
|---|---|---|---|---|
| INCI | m [%] | m [%] | m [%] | m [%] |
| Caprylic/Capric Triglyceride | 4.00 | 4.00 | 4.00 | 4.00 |
| Cetyl Alcohol | 3.00 | 3.00 | 3.00 | 3.00 |
| Aqua + Trisodium EDTA | 1.00 | 1.00 | 1.00 | 1.00 |
| BHT | 0.05 | 0.05 | 0.05 | 0.05 |
| Phenoxyethanol | 0.80 | 0.80 | 0.80 | 0.80 |
| Hydrogenated Coco-Glycerides | 2.00 | 2.00 | 2.00 | 2.00 |
| Glycerol | 7.50 | 7.50 | 7.50 | 7.50 |
| Aqua + Sodium Hydroxide | 0.01 | 0.01 | 0.01 | 0.01 |
| Alcohol Denat. + Aqua | 4.00 | 4.00 | 4.00 | 4.00 |
| Xanthan Gum | 0.30 | 0.30 | 0.30 | 0.30 |
| Acrylates/C10-30 Alkyl Acrylate Crosspolymer | 0.20 | 0.20 | 0.20 | 0.20 |
| Glyceryl Stearate Citrate | 2.00 | 2.00 | 2.00 | 2.00 |
| Dicaprylyl Ether | 4.00 | 4.00 | 4.00 | 4.00 |
| Aqua | to 100 | to 100 | to 100 | to 100 |
| Ac-RFAACAA | 0.25 | - | - | 0.2 |
| GSH | - | 1 | - | - |
| Ac-RFAALAA | - | - | 0.4 | 0.1 |

1-15. (canceled)

16. A peptide, wherein the peptide is effective in the treatment of allergic contact dermatitis caused by urushiols and comprises 2 to 10 amino acids, at least one of these amino acids having one or more reactive side chains.

17. The peptide of claim 16, wherein at least one of the amino acids has one or more free thiol groups.

18. The peptide of claim 16, wherein at least one of the amino acids is histidine, cysteine, lysine, tyrosine, serine, selenocysteine, homocysteine, asparagine, aspartic acid, glutamine, glutamic acid, methionine, phenylalanine or pyrrolysine.

19. The peptide of claim 16, wherein the peptide is GSH, HHHHHH, Ac-RFAACAA, Ac-RFAALAA, RFAALAA, RFAACAA, Ac-RAACAA, RAACAA, Ac-RFACAA, RFACAA, Ac-RFACA or RFACA.

20. A preparation, wherein the preparation is topically applicable and comprises one or more peptides according to claim 16 and is capable of preventing or attenuating allergic reactions caused by urushiols in or on the skin.

21. The preparation of claim 20, wherein at least one of the one or more amino acids of the one or more peptides has one or more free thiol groups.

22. The preparation of claim 20, wherein at least one of the one or more amino acids of the one or more peptides is histidine, cysteine, lysine, tyrosine, serine, selenocysteine, asparagine, aspartic acid, glutamine, glutamic acid, methionine, phenylalanine or pyrrolysine.

23. The preparation of claim 20, wherein at least one of the one or more peptides is GSH, HHHHHH, Ac-RFAACAA, Ac-RFAALAA, RFAALAA, RFAACAA, Ac-RAACAA, RAACAA, Ac-RFACAA, RFACAA, Ac-RFACA or RFACA.

24. The preparation of claim 20, wherein the preparation comprises from 0.01% to 10% by weight of the one or more peptides, based on a total weight of the preparation.

25. The preparation of claim 24, wherein the preparation comprises from 0.1% to 5% by weight of the one or more peptides.

26. The preparation of claim 24, wherein the preparation comprises from 0.2% to 5% by weight of the one or more peptides.

27. The preparation of claim 23, wherein the preparation comprises from 0.01% to 10% by weight of the one or more peptides, based on a total weight of the preparation.

28. The preparation of claim 27, wherein the preparation comprises from 0.1% to 5% by weight of the one or more peptides.

29. The preparation of claim 27, wherein the preparation comprises from 0.2% to 5% by weight of the one or more peptides.

30. The preparation of claim 20, wherein the preparation is present in the form of at least one of an emulsion, a gel or a spray.

31. The preparation of claim 29, wherein the preparation is present in the form of at least one of an emulsion, a gel or a spray.

32. A method of forming a skin barrier against urushiols, wherein the method comprises applying onto the skin the preparation of claim 20.

33. A method of preventing or reducing the penetration and/or accumulation of urushiols on skin and/or in skin, wherein the method comprises applying onto the skin the preparation of claim 20.

34. A method of preventing or attenuating allergic reactions in skin or on skin, wherein the method comprises applying onto the skin the preparation of claim 20.

35. A method of preventing or attenuating allergic contact dermatitis caused by urushiols, wherein the method comprises applying onto skin the preparation of claim 20.
